(19) United States
(12) Patent Application Publication
(10) Pub. No.: US 2021/0343426 A1
(43) Pub. Date: Nov. 4, 2021

(54) Systems and Methods for a Mask with User Health Sensors

(71) Applicant: Ontario Manufacturing and Design Inc., Bracebridge (CA)

(72) Inventor: Robert Andrew Struthers, Bracebridge (CA)

(21) Appl. No.: 17/306,110

(22) Filed: May 3, 2021

Related U.S. Application Data
(60) Provisional application No. 62/704,288, filed on May 2, 2020.

(51) Int. Cl. G16H 50/80; A61B 5/00; G16H 50/20

(52) U.S. Cl. CPC G16H 50/80; A61B 5/0015; A61B 5/0008; A61B 5/6803; G16H 50/20; A61B 5/742; A61B 5/746; A61B 5/7475; A61B 5/7405

(57) ABSTRACT

A computer-implemented method for using a sensor incorporated into a mask, computer program product, and mask with a computer system for obtaining, from a sensor, data associated with a biological attribute of a user. The data may be processed to determine whether the data is within a threshold range. An indication of whether the data is within the threshold range may be provided.

### RELATED CASES

This application claims the benefit of U.S. Provisional Application No. 62/704,288 filed on 2 May 2020, the contents of which are all incorporated by reference.

### BACKGROUND

As society resumes normal activities post Pandemic a need to monitor people's health is very important. Controlling access to airports, theme parks, shopping malls and all other public areas may be critical. Additionally, monitoring employees in high density or high risk commercial environments may keep our society and economy safe. Understanding user health through detecting high core temperatures in real time and responding is critical.

### BRIEF SUMMARY OF DISCLOSURE

In one example implementation, a method, performed by one or more computing devices, may include but is not limited to obtaining, from a sensor, data associated with a biological attribute of a user. The data may be processed to determine whether the data is within a threshold range. An indication of whether the data is within the threshold range may be provided.

One or more of the following example features may be included. Obtaining the data may include receiving a transmission of the data from the sensor. The threshold range may be a temperature range. The indication may be an audible indication. The indication may be a visual indication based upon, at least in part, the threshold range of the data. The visual indication may be one of a color indication and a textual indication. At least one sensor of a plurality of sensors from a plurality of masks may be monitored at a user interface of a computing device and an action may be executed based upon, at least in part, the threshold range of the data.

In another example implementation, a mask with a computing system may include one or more processors and one or more memories configured to perform operations that may include but are not limited to obtaining, from a sensor, data associated with a biological attribute of a user. The data may be processed to determine whether the data is within a threshold range. An indication of whether the data is within the threshold range may be provided.

One or more of the following example features may be included. Obtaining the data may include receiving a transmission of the data from the sensor. The threshold range may be a temperature range. The indication may be an audible indication. The indication may be a visual indication based upon, at least in part, the threshold range of the data. The visual indication may be one of a color indication and a textual indication. At least one sensor of a plurality of sensors from a plurality of masks may be monitored at a user interface of a computing device and an action may be executed based upon, at least in part, the threshold range of the data.

In another example implementation, a computer program product may reside on a computer readable storage medium having a plurality of instructions stored thereon which, when executed across one or more processors, may cause at least a portion of the one or more processors to perform operations for using a sensor incorporated into a mask that may include but are not limited to obtaining, from a sensor, data associated with a biological attribute of a user. The data may be processed to determine whether the data is within a threshold range. An indication of whether the data is within the threshold range may be provided.

One or more of the following example features may be included. Obtaining the data may include receiving a transmission of the data from the sensor. The threshold range may be a temperature range. The indication may be an audible indication. The indication may be a visual indication based upon, at least in part, the threshold range of the data. The visual indication may be one of a color indication and a textual indication. At least one sensor of a plurality of sensors from a plurality of masks may be monitored at a user interface of a computing device and an action may be executed based upon, at least in part, the threshold range of the data.

The details of one or more example implementations are set forth in the accompanying drawings and the description below. Other possible example features and/or possible example advantages will become apparent from the description, the drawings, and the claims. Some implementations may not have those possible example features and/or possible example advantages, and such possible example features and/or possible example advantages may not necessarily be required of some implementations.

### BRIEF DESCRIPTION OF THE DRAWINGS

FIG. 1 is an example diagrammatic view of a sensor process coupled to an example distributed computing network according to one or more example implementations of the disclosure;

FIG. 2 is an example diagrammatic view of a client electronic device of FIG. 1 according to one or more example implementations of the disclosure;

FIG. 3 is an example flowchart of a sensor process according to one or more example implementations of the disclosure;

FIG. 4 is an example diagrammatic view of a mask used with a sensor process according to one or more example implementations of the disclosure; and

FIG. 5 is an example diagrammatic view of a mask device used with a sensor process according to one or more example implementations of the disclosure; and

FIG. 6 is an example diagrammatic view of screen images displayed by a sensor process according to one or more example implementations of the disclosure.

Like reference symbols in the various drawings may indicate like elements.

### DETAILED DESCRIPTION

### System Overview:

In some implementations, the present disclosure may be embodied as a method, system, or computer program product. Accordingly, in some implementations, the present disclosure may take the form of an entirely hardware implementation, an entirely software implementation (including firmware, resident software, micro-code, etc.) or an implementation combining software and hardware aspects that may all generally be referred to herein as a “circuit,” “module” or “system.” Furthermore, in some implementations, the present disclosure may take the form of a computer program product on a computer-usable storage medium having computer-usable program code embodied in the medium.

In some implementations, any suitable computer usable or computer readable medium (or media) may be utilized. The computer readable medium may be a computer readable signal medium or a computer readable storage medium. The computer-usable, or computer-readable, storage medium (including a storage device associated with a computing device or client electronic device) may be, for example, but is not limited to, an electronic, magnetic, optical, electromagnetic, infrared, or semiconductor system, apparatus, device, or any suitable combination of the foregoing. More specific examples (a non-exhaustive list) of the computer-readable medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a digital versatile disk (DVD), a static random access memory (SRAM), a memory stick, a floppy disk, a mechanically encoded device such as punch-cards or raised structures in a groove having instructions recorded thereon, a media such as those supporting the internet or an intranet, or a magnetic storage device. Note that the computer-usable or computer-readable medium could even be a suitable medium upon which the program is stored, scanned, compiled, interpreted, or otherwise processed in a suitable manner, if necessary, and then stored in a computer memory. In the context of the present disclosure, a computer-usable or computer-readable, storage medium may be any tangible medium that can contain or store a program for use by or in connection with the instruction execution system, apparatus, or device.

In some implementations, a computer readable signal medium may include a propagated data signal with computer readable program code embodied therein, for example, in baseband or as part of a carrier wave. In some implementations, such a propagated signal may take any of a variety of forms, including, but not limited to, electro-magnetic, optical, or any suitable combination thereof. In some implementations, the computer readable program code may be transmitted using any appropriate medium, including but not limited to the internet, wireline, optical fiber cable, RF, etc. In some implementations, a computer readable signal medium may be any computer readable medium that is not a computer readable storage medium and that can communicate, propagate, or transport a program for use by or in connection with an instruction execution system, apparatus, or device.

In some implementations, computer program code for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java®, Smalltalk, C++ or the like. Java® and all Java-based trademarks and logos are trademarks or registered trademarks of Oracle and/or its affiliates. However, the computer program code for carrying out operations of the present disclosure may also be written in conventional procedural programming languages, such as the “C” programming language, PASCAL, or similar programming languages, as well as in scripting languages such as Javascript, PERL, or Python. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through a local area network (LAN), a wide area network (WAN), a body area network BAN), a personal area network (PAN), a metropolitan area network (MAN), etc., or the connection may be made to an external computer (for example, through the internet using an Internet Service Provider). In some implementations, electronic circuitry including, for example, programmable logic circuitry, an application specific integrated circuit (ASIC), field-programmable gate arrays (FPGAs) or other hardware accelerators, micro-controller units (MCUs), or programmable logic arrays (PLAs) may execute the computer readable program instructions/code by utilizing state information of the computer readable program instructions to personalize the electronic circuitry, in order to perform aspects of the present disclosure.

In some implementations, the flowchart and block diagrams in the figures illustrate the architecture, functionality, and operation of possible implementations of apparatus (systems), methods and computer program products according to various implementations of the present disclosure. Each block in the flowchart and/or block diagrams, and combinations of blocks in the flowchart and/or block diagrams, may represent a module, segment, or portion of code, which comprises one or more executable computer program instructions for implementing the specified logical function(s)/act(s). These computer program instructions may be provided to a processor of a general purpose computer, special purpose computer, or other programmable data processing apparatus to produce a machine, such that the computer program instructions, which may execute via the processor of the computer or other programmable data processing apparatus, create the ability to implement one or more of the functions/acts specified in the flowchart and/or block diagram block or blocks or combinations thereof. It should be noted that, in some implementations, the functions noted in the block(s) may occur out of the order noted in the figures (or combined or omitted). For example, two blocks shown in succession may, in fact, be executed substantially concurrently, or the blocks may sometimes be executed in the reverse order, depending upon the functionality involved.

In some implementations, these computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks or combinations thereof.

In some implementations, the computer program instructions may also be loaded onto a computer or other programmable data processing apparatus to cause a series of operational steps to be performed (not necessarily in a particular order) on the computer or other programmable apparatus to produce a computer implemented process such that the instructions which execute on the computer or other programmable apparatus provide steps for implementing the functions/acts (not necessarily in a particular order) specified in the flowchart and/or block diagram block or blocks or combinations thereof.

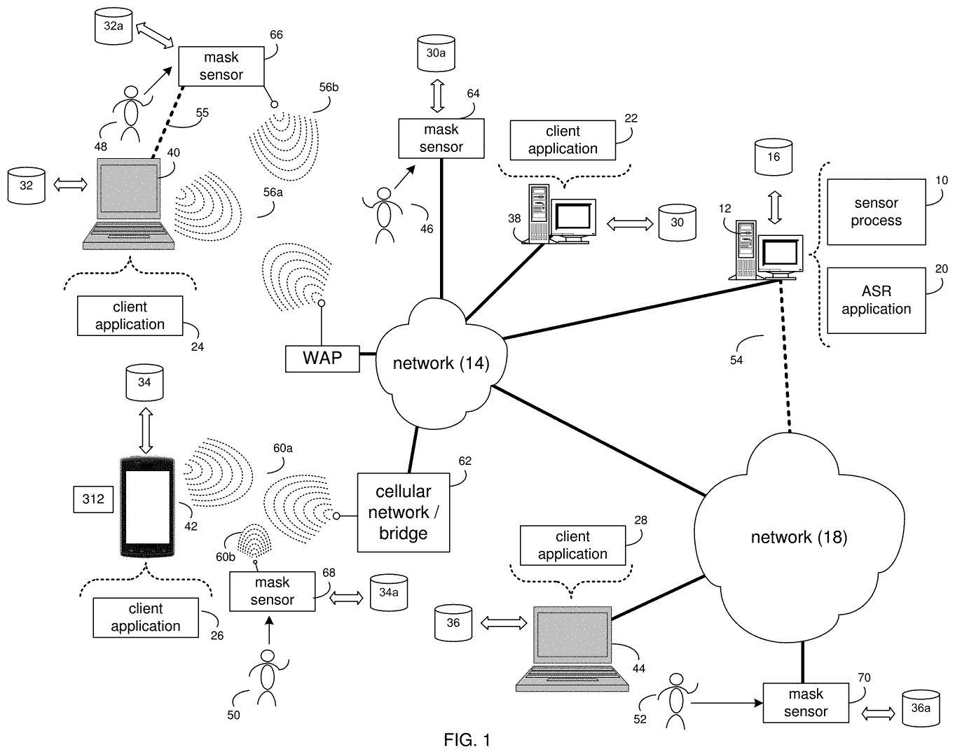

Referring now to the example implementation of FIG. 1, there is shown sensor process 10 that may reside on and may be executed by a computer (e.g., computer 12), which may be connected to a network (e.g., network 14) (e.g., the internet or a local area network). Examples of computer 12 (and/or one or more of the client electronic devices noted below) may include, but are not limited to, a storage system (e.g., a Network Attached Storage (NAS) system, a Storage Area Network (SAN)), a personal computer(s), a laptop computer(s), mobile computing device(s), a server computer, a series of server computers, a mainframe computer(s), or a computing cloud(s). A SAN may include one or more of the client electronic devices, including a RAID device and a NAS system. In some implementations, each of the aforementioned may be generally described as a computing device. In certain implementations, a computing device may be a physical or virtual device. In many implementations, a computing device may be any device capable of performing operations, such as a dedicated processor, a portion of a processor, a virtual processor, a portion of a virtual processor, portion of a virtual device, or a virtual device. In some implementations, a processor may be a physical processor or a virtual processor. In some implementations, a virtual processor may correspond to one or more parts of one or more physical processors. In some implementations, the instructions/logic may be distributed and executed across one or more processors, virtual or physical, to execute the instructions/logic. Computer 12 may execute an operating system, for example, but not limited to, Microsoft® Windows®; Mac® OS X®; Red Hat® Linux®, Windows® Mobile, Chrome OS, Blackberry OS, Fire OS, or a custom operating system. (Microsoft and Windows are registered trademarks of Microsoft Corporation in the United States, other countries or both; Mac and OS X are registered trademarks of Apple Inc. in the United States, other countries or both; Red Hat is a registered trademark of Red Hat Corporation in the United States, other countries or both; and Linux is a registered trademark of Linus Torvalds in the United States, other countries or both).

In some implementations, as will be discussed below in greater detail, a sensor process, such as sensor process 10 of FIG. 1, may obtain, from a sensor, data associated with a biological attribute of a user. The data may be processed to determine whether the data is within a threshold range. An indication of whether the data is within the threshold range may be provided.

In some implementations, the instruction sets and subroutines of sensor process 10, which may be stored on storage device, such as storage device 16, coupled to computer 12, may be executed by one or more processors and one or more memory architectures included within computer 12. In some implementations, storage device 16 may include but is not limited to: a hard disk drive; all forms of flash memory storage devices; a tape drive; an optical drive; a RAID array (or other array); a random access memory (RAM); a read-only memory (ROM); or combination thereof. In some implementations, storage device 16 may be organized as an extent, an extent pool, a RAID extent (e.g., an example 4D+1P R5, where the RAID extent may include, e.g., five storage device extents that may be allocated from, e.g., five different storage devices), a mapped RAID (e.g., a collection of RAID extents), or combination thereof.

In some implementations, network 14 may be connected to one or more secondary networks (e.g., network 18), examples of which may include but are not limited to: a local area network; a wide area network or other telecommunications network facility; or an intranet, for example. The phrase “telecommunications network facility,” as used herein, may refer to a facility configured to transmit, and/or receive transmissions to/from one or more mobile client electronic devices (e.g., cellphones, etc.) as well as many others.

In some implementations, computer 12 may include a data store, such as a database (e.g., relational database, object-oriented database, triplestore database, etc.) and may be located within any suitable memory location, such as storage device 16 coupled to computer 12. In some implementations, data, metadata, information, etc. described throughout the present disclosure may be stored in the data store. In some implementations, computer 12 may utilize any known database management system such as, but not limited to, DB2, in order to provide multi-user access to one or more databases, such as the above noted relational database. In some implementations, the data store may also be a custom database, such as, for example, a flat file database or an XML database. In some implementations, any other form(s) of a data storage structure and/or organization may also be used. In some implementations, sensor process 10 may be a component of the data store, a standalone application that interfaces with the above noted data store and/or an applet/application that is accessed via client applications 22, 24, 26, 28. In some implementations, the above noted data store may be, in whole or in part, distributed in a cloud computing topology. In this way, computer 12 and storage device 16 may refer to multiple devices, which may also be distributed throughout the network. An example cloud computing environment that may be used with the disclosure may include but is not limited to, e.g., Elastic Cloud Storage™ from Dell EMC™ of Hopkinton, Mass. In some implementations, other cloud computing environments may be used without departing from the scope of the disclosure.

In some implementations, computer 12 may execute an automatic speech recognition application (e.g., automatic speech recognition application 20), examples of which may include, but are not limited to, e.g., an automatic speech recognition (ASR) application (e.g., automatic speech recognition application 20), examples of which may include, but are not limited to, e.g., an automatic speech recognition (ASR) application (e.g., modeling, etc.), a natural language understanding (NLU) application (e.g., machine learning, intent discovery, etc.), a text to speech (TTS) application (e.g., context awareness, learning, etc.), a speech signal enhancement (SSE) application (e.g., multi-zone processing/beamforming, noise suppression, etc.), a voice biometrics/wake-up-word processing application, a web conferencing application, a video conferencing application, a voice-over-IP application, a video-over-IP application, an Instant Messaging (IM)/“chat” application, a short messaging service (SMS)/multimedia messaging service (MMS) application, or other application that allows for virtual meeting and/or remote collaboration. In some implementations, sensor process 10 and/or automatic speech recognition application 20 may be accessed via one or more of client applications 22, 24, 26, 28. In some implementations, sensor process 10 may be a standalone application, or may be an applet/application/script/extension that may interact with and/or be executed within automatic speech recognition application 20, a component of automatic speech recognition application 20, and/or one or more of client applications 22, 24, 26, 28. In some implementations, automatic speech recognition application 20 may be a standalone application, or may be an applet/application/script/extension that may interact with and/or be executed within sensor process 10, a component of sensor process 10, and/or one or more of client applications 22, 24, 26, 28. In some implementations, one or more of client applications 22, 24, 26, 28 may be a standalone application, or may be an applet/application/script/extension that may interact with and/or be executed within and/or be a component of sensor process 10 and/or automatic speech recognition application 20. Examples of client applications 22, 24, 26, 28 may include, but are not limited to, e.g., a web conferencing application, a video conferencing application, a voice-over-IP application, a video-over-IP application, an Instant Messaging (IM)/“chat” application, a short messaging service (SMS)/multimedia messaging service (MMS) application, or other application that allows for virtual meeting and/or remote collaboration, a mask sensor application, a standard and/or mobile web browser, an email application (e.g., an email client application), a textual and/or a graphical user interface, a customized web browser, a plugin, an Application Programming Interface (API), or a custom application. The instruction sets and subroutines of client applications 22, 24, 26, 28, which may be stored on storage devices 30, 32, 34, 36, 30a, 32a, 34a, 36a, coupled to client electronic devices 38, 40, 42, 44 and/or mask sensor 64, 66, 68, 70, may be executed by one or more processors and one or more memory architectures incorporated into client electronic devices 38, 40, 42, 44 and/or mask sensor 64, 66, 68, 70.

In some implementations, one or more of storage devices 30, 32, 34, 36, 30a, 32a, 34a, 36a, may include but are not limited to: hard disk drives; flash drives, tape drives; optical drives; RAID arrays; random access memories (RAM); and read-only memories (ROM). Examples of client electronic devices 38, 40, 42, 44 (and/or computer 12) may include, but are not limited to, a personal computer (e.g., client electronic device 38), a laptop computer (e.g., client electronic device 40), a smart/data-enabled, cellular phone (e.g., client electronic device 42), a notebook computer (e.g., client electronic device 44), a tablet, a server, a television, a smart television, a smart speaker, an Internet of Things (IoT) device, a media (e.g., audio/video, photo, etc.) capturing and/or output device, a mask device, an audio input and/or recording device (e.g., a handheld microphone, a lapel microphone, an embedded microphone (such as those embedded within eyeglasses, smart phones, tablet computers and/or watches, etc.), and a dedicated network device. Additionally/alternatively, one or more of client electronic devices 38, 40, 42, 44 may include a mask with one or more sensors (e.g., mask sensor 64, 66, 68, 70). Client electronic devices 38, 40, 42, 44 may each execute an operating system, examples of which may include but are not limited to, Android™, Apple® iOS®, Mac® OS X®; Red Hat® Linux®, Windows® Mobile, Chrome OS, Blackberry OS, Fire OS, or a custom operating system.

In some implementations, one or more of client applications 22, 24, 26, 28 may be configured to effectuate some or all of the functionality of sensor process 10 (and vice versa). Accordingly, in some implementations, sensor process 10 may be a purely server-side application, a purely client-side application, or a hybrid server-side/client-side application that is cooperatively executed by one or more of client applications 22, 24, 26, 28 and/or sensor process 10.

In some implementations, one or more of client applications 22, 24, 26, 28 may be configured to effectuate some or all of the functionality of automatic speech recognition application 20 (and vice versa). Accordingly, in some implementations, automatic speech recognition application 20 may be a purely server-side application, a purely client-side application, or a hybrid server-side/client-side application that is cooperatively executed by one or more of client applications 22, 24, 26, 28 and/or automatic speech recognition application 20. As one or more of client applications 22, 24, 26, 28, sensor process 10, and automatic speech recognition application 20, taken singly or in any combination, may effectuate some or all of the same functionality, any description of effectuating such functionality via one or more of client applications 22, 24, 26, 28, sensor process 10, automatic speech recognition application 20, or combination thereof, and any described interaction(s) between one or more of client applications 22, 24, 26, 28, sensor process 10, automatic speech recognition application 20, or combination thereof to effectuate such functionality, should be taken as an example only and not to limit the scope of the disclosure.

In some implementations, one or more of users 46, 48, 50, 52 and/or one or more of mask sensor 64, 66, 68, 70 may access computer 12 and sensor process 10 (e.g., using one or more of client electronic devices 38, 40, 42, 44) directly through network 14 or through secondary network 18. Further, computer 12 may be connected to network 14 through secondary network 18, as illustrated with phantom link line 54. Sensor process 10 may include one or more user interfaces, such as browsers and textual or graphical user interfaces, through which users 46, 48, 50, 52 may access sensor process 10 and/or mask sensor 64, 66, 68, 70.

In some implementations, one or more of the various client electronic devices and/or one or more of mask sensor 64, 66, 68, 70 may be directly or indirectly coupled to network 14 (or network 18). For example, personal computer 38 and mask sensor 64 are shown directly coupled to network 14 via a hardwired network connection. Further, notebook computer 44 and mask sensor 70 are shown directly coupled to network 18 via a hardwired network connection. Laptop computer 40 and mask sensor 66 are shown wirelessly coupled to network 14 via wireless communication channels 56a and 56b respectively established between laptop computer 40 and wireless access point (i.e., WAP) 58 and between mask sensor 66 and WAP 58, which is shown directly coupled to network 14. WAP 58 may be, for example, an IEEE 802.11a, 802.11b, 802.11g, Wi-Fi®, RFID, and/or Bluetooth™ (including Bluetooth™ Low Energy) device that is capable of establishing wireless communication channel 56a between laptop computer 40 and WAP 58 and wireless communication channel 56b between mask sensor 66 and WAP 58. Additionally/alternatively, a mask sensor (e.g., DEVICE 66) may be directly (and/or wirelessly) coupled to a client electronic device (e.g., client electronic device 40) as illustrated with phantom link line 55. Thus, information may be communicated from a mask sensor (e.g., mask sensor 66) to a client electronic device (e.g., client electronic device 40), where the information may be communicated, e.g., to computer 12 via, e.g., a network (e.g., network 14). Smart phone 42 and mask sensor 68 are shown wirelessly coupled to network 14 via wireless communication channels 60a and 60b respectively established between smart phone 42 and cellular network/bridge 62 and mask sensor 68 and cellular network/bridge 62, which is shown by example directly coupled to network 14.

In some implementations, some or all of the IEEE 802.11x specifications may use Ethernet protocol and carrier sense multiple access with collision avoidance (i.e., CSMA/CA) for path sharing. The various 802.11x specifications may use phase-shift keying (i.e., PSK) modulation or complementary code keying (i.e., CCK) modulation, for example. Bluetooth™ (including Bluetooth™ Low Energy) is a telecommunications industry specification that allows, e.g., mobile phones, computers, smart phones, and other electronic devices (e.g., mask sensor 64, 66, 68, 70) to be interconnected using a short-range wireless connection. Other forms of interconnection (e.g., Near Field Communication (NFC)) may also be used.

In some implementations, various I/O requests (e.g., I/O request 15) may be sent from, e.g., client applications 22, 24, 26, 28 and/or mask sensor 64, 66, 68, 70 to, e.g., computer 12 (and vice versa). Examples of I/O request 15 may include but are not limited to, data write requests (e.g., a request that content be written to computer 12) and data read requests (e.g., a request that content be read from computer 12).

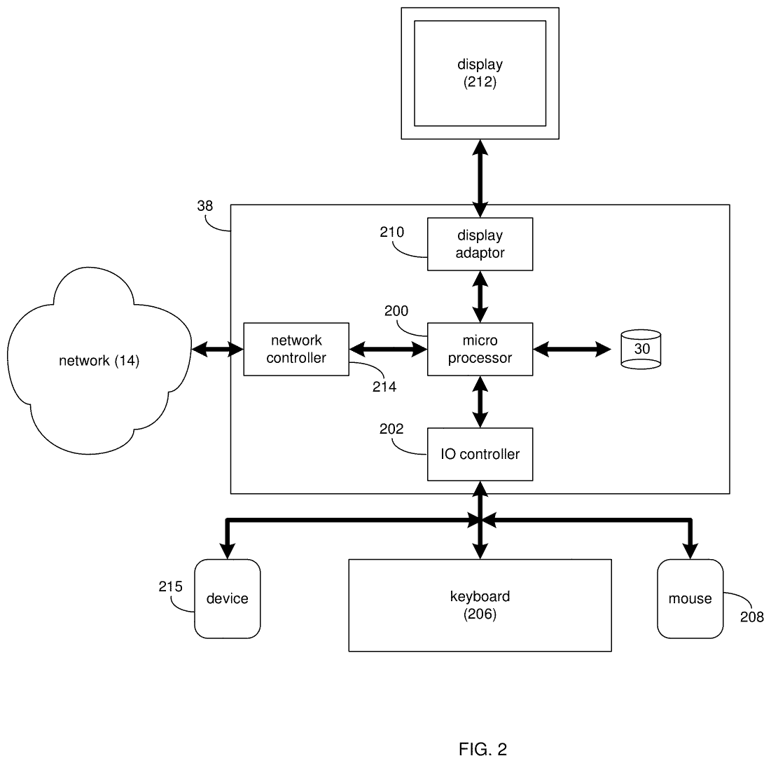

Referring also to the example implementation of FIG. 2, there is shown a diagrammatic view of client electronic device 38. While client electronic device 38 is shown in this figure, this is for example purposes only and is not intended to be a limitation of this disclosure, as other configurations are possible. Additionally, any computing device capable of executing, in whole or in part, sensor process 10 may be substituted for client electronic device 38 (in whole or in part) within FIG. 2, examples of which may include but are not limited to computer 12 and/or one or more of client electronic devices 38, 40, 42, 44 and/or one or more of mask sensor 64, 66, 68, 70.

In some implementations, client electronic device 38 may include a processor (e.g., microprocessor 200) configured to, e.g., process data and execute the above-noted code/instruction sets and subroutines. Microprocessor 200 may be coupled via a storage adaptor to the above-noted storage device(s) (e.g., storage device 30). An I/O controller (e.g., I/O controller 202) may be configured to couple microprocessor 200 with various devices (e.g., via wired or wireless connection), such as keyboard 206, pointing/selecting device (e.g., touchpad, touchscreen, mouse 208, etc.), custom device (e.g., device 215, which in some implementations may be a mask sensor as discussed throughout), USB ports, and printer ports. A display adaptor (e.g., display adaptor 210) may be configured to couple display 212 (e.g., touchscreen monitor(s), plasma, CRT, or LCD monitor(s), etc.) with microprocessor 200, while network controller/adaptor 214 (e.g., an Ethernet adaptor) may be configured to couple microprocessor 200 to the above-noted network 14 (e.g., the Internet or a local area network).

### The Sensor Process:

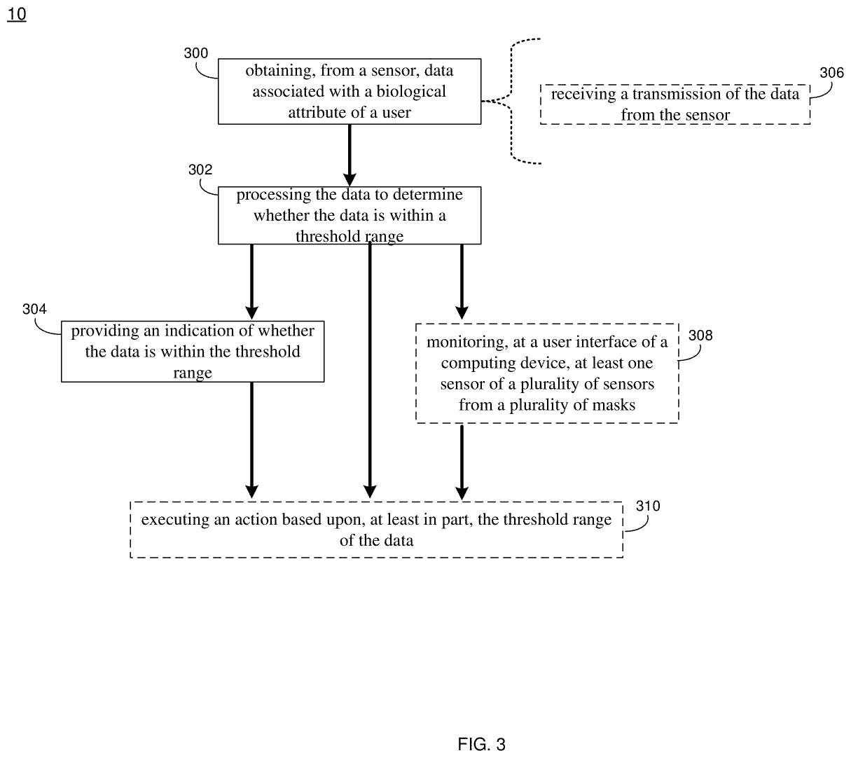

As discussed above and referring also at least to the example implementations of FIGS. 3-6, sensor process 10 may obtain 300, from a sensor, data associated with a biological attribute of a user. Sensor Process 10 may process 302 the data to determine whether the data is within a threshold range. Sensor Process 10 may provide 304 an indication of whether the data is within the threshold range.

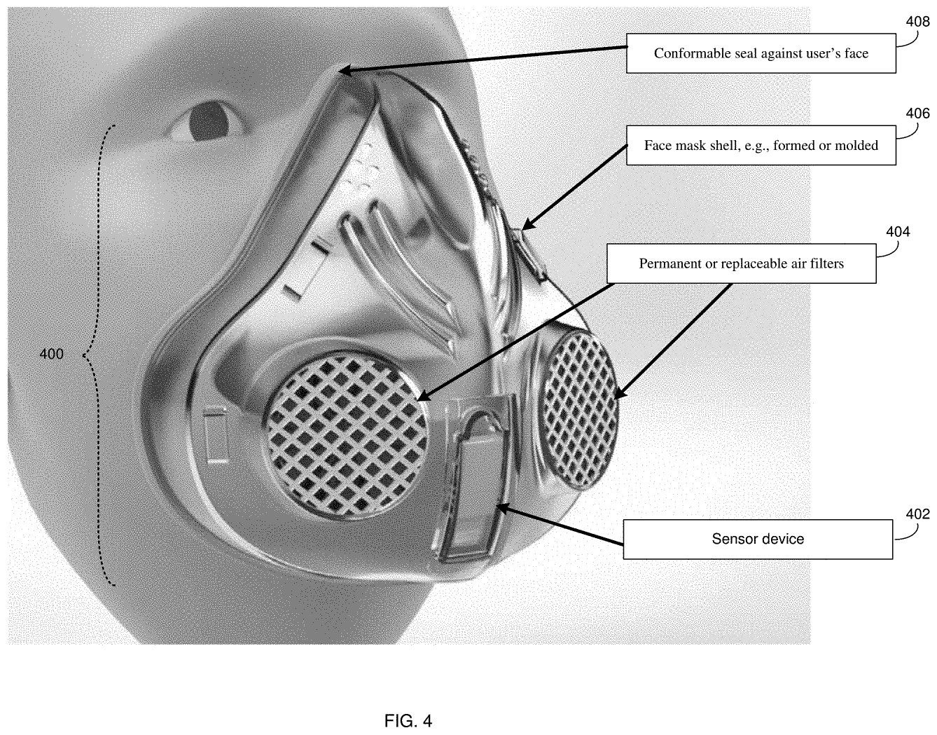

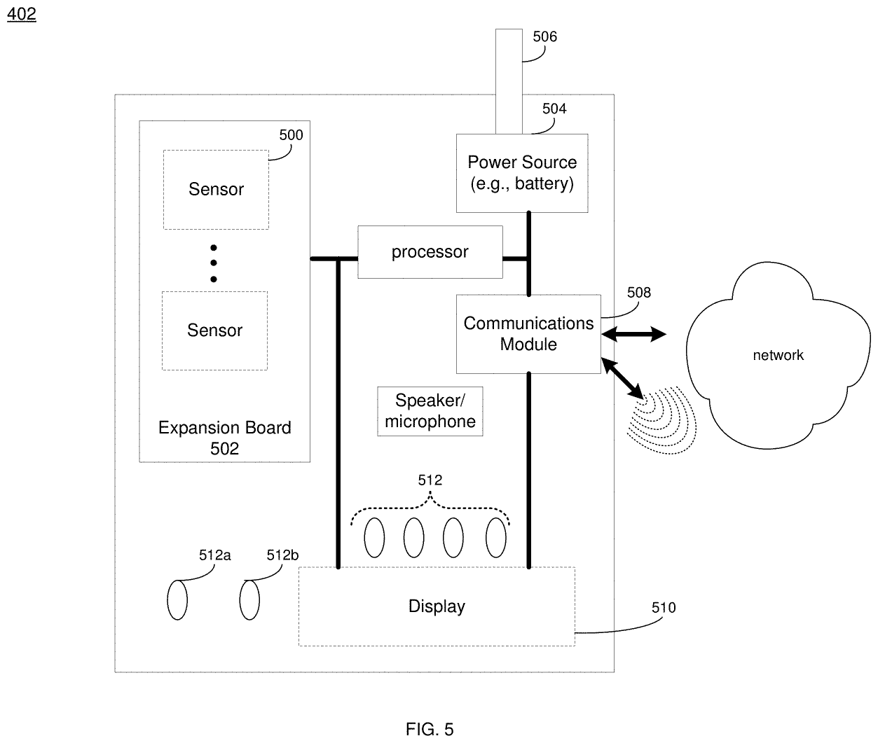

In some implementations, sensor process 10 may obtain 300, from a sensor, data associated with a biological attribute of a user. For instance, and referring to the example implementation of FIGS. 4-5, an example mask 400 (e.g., mask 400) with a sensor device (e.g., sensor device 402, which may include the above-noted mask sensors such as mask sensor 68) is shown. As used herein, the terms mask sensor and sensor device may be used interchangeably. In some implementations, mask 400 may include an adjustable head strap to hold the mask on the user's head. In the example, as will be discussed in greater detail below, mask 400 may include sensor device 402, air filters 404 (e.g., permanent or replaceable air filters), a face mask shell 406 (e.g., formed or molded), and a seal (e.g., seal 408) that may rest comfortably against the user's face (e.g., user 50). In some implementations, face mask shell 406 may be a clear mask shell to allow the mouth of the user to be seen, e.g., to help with lip reading for the disabled or to help pick up on social mannerisms (e.g., smiling). In some implementations, seal 408 may include a skin safe gasket (e.g., silicone or similar material) to seal against the face which may be reusable and held on with a strap around the users head. This may allow for a consistent seal across a large range of face sizes and shapes. By manufacturing the mask out of plastic, the amount of filter material may be greatly reduced and mask 400 may be sterilized for reuse. However, it will be appreciated that disposable masks may also be used without departing from the present disclosure. In some implementations, shell 406 may be a thermoformed, vacuum-formed or molded shell covering the mouth and nose which may be disposable or reusable.

Sensor device 402 may include, for example, one or more sensors (e.g., sensor 500) that may be located on one or more expansion boards (e.g., expansion board 502). In some implementations, sensor 500 may include one or more sensors to detect ambient temperature in the mask (either from the user's breath in the mask or skin contact). For example, the front or back side of sensor device 402 may include an opening to allow for airflow and optic measurements from the user, and in some implementations, skin temperature may be measured (e.g., by an IR sensor or other type of temperature sensor)) and calibrated by an environmental air sensor. Sensor 500 may be mounted on the outside of mask 400, and may include an IR sensor for measuring the body temperature which may be calibrated to point in the users nose or mouth. However, other temperature sensors, as well as other types of sensors may additionally/alternatively be used to detect the following, e.g.,: air pressure inside the mask for exhalation pressure and filter monitoring status, lead, volatile organic compounds (VOCs), gas levels (e.g., CO, CO₂, O₂), ambient pressure, humidity, Ultraviolet (UV), global positioning via a global positioning system sensor, respiratory rate, blood alcohol level, delta-9-tetrahydrocannabinol (THC) levels, biological analytes (e.g., bacterial, viruses, mold, proteins, etc.), pH levels, sound (e.g., via ASR application 20 and microphones/speakers to receive and/or amplify the voice of the person wearing mask 400 or issue/receive voice commands/music to/from a mobile computing device), or any other sensor capable of being incorporated into mask 400. As such, the use of a temperature sensor should be taken as example only and not to otherwise limit the scope of the present disclosure. Additionally, while the sensors are described as being electronic sensors, it will be appreciated that non-electronic sensors may also be used without departing from the scope of the present disclosure (e.g., litmus paper sensors or other lateral flow assays). As such, the description of electronic sensors should be taken as example only and not to otherwise limit the scope of the present disclosure.

In some implementations, expansion board 502 may be used to handle one or more of the above-noted sensors (simultaneously or individually), which may be interchangeable by the user or manufacturer in some implementations. For example, expansion board may include solder pads on a PCB that may be utilized to allow for different models to be manufactured to monitor different chemicals and/or environmental conditions via sensor 500. In some implementations, the PCB of expansion board 502 may have a pad with, e.g., up to 8 pins attaching to a microprocessor (such as the processor discussed above). Sensor 500 may be attached to as many of the required pins as needed and the firmware may be updated to read that sensor. For example, a lead sensor may require 2 pins, VOC may require 4 pins, UV may require 2 pins, etc. It will be appreciated that a single sensor may read multiple attributes, such as an example BME680 sensor from Bosch, which may read VOC, humidity, ambient temperature and ambient air pressure. Sensor 502 may also be used to determine filter life as air pressure changes once calibrated for the specific type of mask. In some implementations, an optional press fit socket may be used on these solders pads, enabling the functionality for a user to interchange sensors as desired. After unplugging a sensor and plugging a new one in, sensor device 402 may automatically detect and enable the new sensor, and/or the user may use a user interface (e.g., via client application 26) to select the sensor to enable it.

In some implementations, sensor device 402 may be attached or otherwise embedded in mask 400 (or other personal protective equipment (PPE)) using any number of techniques. For example, sensor device 402 may be attached or otherwise embedded in mask 400 or other PPE via, e.g., a permanent or temporary adhesive, hook and loop type fastener, a magnet and receiver/second magnet, a static cling fastener, a mechanical clip, a mechanical clip utilizing a plastic tab, a mechanical fit utilizing friction against surrounding components, elastic or elastomer pocket, screws or bolts or sewing, a housing molded directly to the mask or other PPE, etc.

In some implementations, sensor device 402 may include a power source (e.g., power source 504 which may include but is not limited to a rechargeable battery that is rechargeable via a charging port, such as charging port 506 (e.g., USB, Micro-USB, wireless (e.g., induction charging), or similar charging techniques), or a non-rechargeable battery). In some implementations, power source 504 may be charged using solar technology, with a battery being used as a backup power source.

In some implementations, sensor device 402 may include a communications module (e.g., communications module 508). For instance, as will be discussed below, communications module 508 may include any of the above-noted wired/wireless communication techniques (e.g., Bluetooth™, NFC, Ethernet, etc.) to receive and/or transmit data (e.g., sensor data or any other types of I/O data) from/to a computing device (e.g., computer 12, client electronic device 42, etc.).

In some implementations, sensor device 402 may include one or more visual displays (e.g., display 510 and/or light emitting diodes (LEDs) 512). For instance, display 510 may be an LCD display, capable of displaying textual data (e.g., letters, numbers) as well as other colored or black/white designs. Other types of displays may also be used without departing from the scope of the disclosure. In some implementations, LEDs 512 (as well as LEDs 512a and 512b) may be capable of displaying single or multiple colors. For example, LED 512a may be a green LED, whereas LED 512b may be a red LED. Conversely, LED 512a may be both capable of being a red and green LED, as well as any other color. LEDs 512 (including LEDs 512a and 512b) as well as display 510 may be capable of providing visual alerts, such as the data obtained from sensor 500 (e.g., temperature), connectivity alerts/levels (e.g., 4 blue LEDs for LEDs 512 may be used for communications signal strength, or to show rate of change in the sensor status), low battery alerts/levels, etc. In some implementations, display 510 may be customized by the user to display temperature, sensor status, or a custom message such as the users name or instructions how to proceed in the event of a detected change in safety of the environment/user (e.g., detection of lead or increased temperature of the user), which may be customized by the user via the user interface (e.g., via client application 26). The functionality of the above-noted LEDs may also be customized via the user interface.

It will be appreciated that the configuration of mask 400 and/or sensor device 402 may vary without departing from the scope of the present disclosure. As such, the particular configuration shown in FIGS. 4-5 may include more or less elements, as well as varying locations of elements without departing from the scope of the present disclosure. In some implementations, mask 400 may include, e.g., N95 masks, however, any other mask or face covering capable of incorporating sensor device 402 may be used without departing from the scope of the present disclosure. For example, mask 400 may also be considered a face shield as used herein.

As noted above, sensor process 10 may obtain 300 (e.g., from sensor 500), data associated with a biological attribute of a user. For instance, assume for example purposes only that sensor 500 is a temperature sensor. In the example, by wearing mask 400 with sensor device 402, sensor process 10 may obtain 300 a biological attribute (e.g., temperature) of user 50, as described above. In some implementations, obtaining 300 the data may include receiving 306 a transmission of the data from the sensor. For instance, the data obtained 300 from sensor 500 may be received 306 by other components of sensor device 402 (e.g., processor, communications module 508, display 510, LEDs, etc.). Additionally/alternatively, as will be discussed further below, the data obtained 300 from sensor 500 may be transmitted (e.g., via communications module 508) from sensor device 402 and received 306 by, e.g., computer 12 or any of the above-noted client electronic devices.

In some implementations, sensor process 10 may process 302 the data to determine whether the data is within a threshold range. For instance, assume for example purposes only that, in some implementations, the threshold range may be a temperature range. In the example, the temperature data obtained 300 by sensor 500 may be processed 302 to determine whether the temperature is within a threshold range. For instance, assume for example purposes only that the temperature range of a healthy person is between 97° F. (36.1° C.) and 99° F. (37.2° C.). In the example, sensor process 10 may process 302 the temperature data to determine whether it is within the threshold range of 97° F. (36.1° C.) and 99° F. (37.2° C.). It is noted that the threshold range may vary depending on what data is being collected (e.g., a humidity threshold range for humidity data).

In some implementations, sensor process 10 may provide 304 an indication of whether the data is within the threshold range. For instance, in some implementations, the indication may be an audible indication. For example, if the temperature of the user is 98.6° F. (and thus within the above-noted threshold range), there may be no audible indication (noise). Conversely, if the temperature of the user is 99.1° F. (and thus outside of the above-noted threshold range), there may be an audible indication (noise). The noise (e.g., continuous tone or verbal message) may come from a speaker that is part of sensor device 402, or the noise may come from a remote device (e.g., client electronic device 50) after receiving an indication that the temperature is outside of the above-noted threshold range.

In some implementations, the indication may be a visual indication based upon, at least in part, the threshold range of the data, and in some implementations, the visual indication may be one of a color indication and a textual indication. For example, if the temperature of the user is 98.6° F. (and thus within the above-noted threshold range indicating no fever with a healthy core temperature), LED 512a on the front of mask 400 may be green and LED 512b may be off. Conversely, if the temperature of the user is 99.1° F. (and thus outside of the above-noted threshold range indicating a possible fever and something wrong with the user's health), LED 512b may be red and LED 512a may be off. Various combinations of colors could also be used. For instance, both LED 512a and 512b could be green or red depending on the temperature, or a single LED 512a may be used for both colors depending on the temperature, thus negating the need for LED 512b. In some implementations, one or more of the LEDs may turn yellow if the temperature is approaching the upper/lower limits of the threshold range, or if there is a detected electronic fault (e.g., error or low battery) or if communication module 508 is disconnected or otherwise out of range from the network/client electronic device. It will be appreciated that various other color schemes could also be used without departing from the scope of the disclosure. In some implementations, display 510 may display the temperature, and may also use the above-noted red/yellow/green color scheme to do so depending on the temperature.

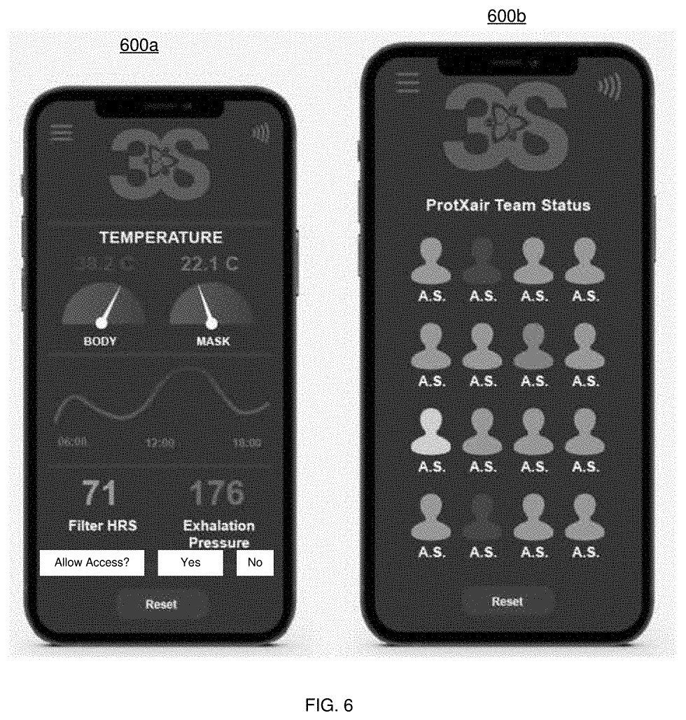

In some implementations, sensor process 10 may, e.g., at a user interface of a computing device, monitor 308 at least one sensor of a plurality of sensors from a plurality of masks and may execute 310 an action based upon, at least in part, the threshold range of the data. For instance, and referring at least to the example implementation of FIG. 6, an example user interface (e.g., user interface 600a and 600b) is shown. User interface 600b is an example user interface that is receiving 306 data from a plurality of masks/users, and is thus being monitored 308. In the example, each user may be displayed on user interface 600b using a similar color scheme as noted above with the LEDs (e.g., green, yellow, red) according to the temperature of the individual user (or possible fault with the device as noted above with regard to the color yellow). User interface 600a is an example user interface with only one mask/user being monitored/displayed, which may be displayed as a result of a user (e.g., administrator) selecting a particular user from user interface 600b. User interface 600a may display, e.g., body temperature, mask temperature, trend graph showing rate of temperature change and time of each temperature taken at certain intervals or temperature thresholds, filter hours and exhalation pressure based on when they reset the filters, etc. It will be appreciated that more or less elements may be monitored and displayed by user interface 600a/600b and in a different customized format without departing from the scope of the present disclosure. As such, the example user interface 600a/600b should be taken as example only and not to otherwise limit the scope of the present disclosure. Moreover, it will be appreciated that should the type of sensor change, what will be displayed will also change based upon the type of sensor added.

In some implementations, each of the masks/users may be monitored by a central monitoring station (which may be made available through a secure web platform), as well as by a client application on a user's individual client electronic device. In some implementations, user interface 600a/600b may be used with various security features. For instance, assume for example purposes only that user interface 600a includes the option to allow or deny access to, e.g., a security gate. In the example, sensor process 10 may automatically execute 310 the action of allowing access through the gate if the temperature of the user attempting to get through the gate is determined to be 98.6° F. (and thus within the above-noted threshold range indicating no fever with a healthy core temperature). Conversely, sensor process 10 may automatically execute 310 the action of denying access through the gate if the temperature of the user attempting to get through the gate is determined to be 99.1° F. (and thus outside of the above-noted threshold range indicating a possible fever and something wrong with the user's health). In some implementations, access may also be granted or denied manually (e.g., via the user selecting the example and non-limiting “Yes” “No” icons on user interface 600a). It will be appreciated that the sensor data, as well as other user status, may be transmitted (e.g., via communications module 508) to monitoring stations, access gates, employers, or any other authority that may need to understand user health.

The terminology used herein is for the purpose of describing particular implementations only and is not intended to be limiting of the disclosure. As used herein, the singular forms “a”, “an” and “the” are intended to include the plural forms as well, unless the context clearly indicates otherwise. As used herein, the language “at least one of A, B, and C” (and the like) should be interpreted as covering only A, only B, only C, or any combination of the three, unless the context clearly indicates otherwise. It will be further understood that the terms “comprises” and/or “comprising,” when used in this specification, specify the presence of stated features, integers, steps (not necessarily in a particular order), operations, elements, and/or components, but do not preclude the presence or addition of one or more other features, integers, steps (not necessarily in a particular order), operations, elements, components, and/or groups thereof.

The corresponding structures, materials, acts, and equivalents (e.g., of all means or step plus function elements) that may be in the claims below are intended to include any structure, material, or act for performing the function in combination with other claimed elements as specifically claimed. The description of the present disclosure has been presented for purposes of illustration and description, but is not intended to be exhaustive or limited to the disclosure in the form disclosed. Many modifications, variations, substitutions, and any combinations thereof will be apparent to those of ordinary skill in the art without departing from the scope and spirit of the disclosure. The implementation(s) were chosen and described in order to explain the principles of the disclosure and the practical application, and to enable others of ordinary skill in the art to understand the disclosure for various implementation(s) with various modifications and/or any combinations of implementation(s) as are suited to the particular use contemplated.

Having thus described the disclosure of the present application in detail and by reference to implementation(s) thereof, it will be apparent that modifications, variations, and any combinations of implementation(s) (including any modifications, variations, substitutions, and combinations thereof) are possible without departing from the scope of the disclosure defined in the appended claims.

What is claimed is:

1. A computer-implemented method for using a sensor incorporated into a mask comprising:
obtaining, from a sensor, data associated with a biological attribute of a user;
processing the data to determine whether the data is within a threshold range; and
providing an indication of whether the data is within the threshold range.

2. The computer-implemented method of claim 1 wherein obtaining the data includes receiving a transmission of the data from the sensor.

3. The computer-implemented method of claim 1 wherein the threshold range is a temperature range.

4. The computer-implemented method of claim 1 wherein the indication an audible indication.

5. The computer-implemented method of claim 1 wherein the indication a visual indication based upon, at least in part, the threshold range of the data.

6. The computer-implemented method of claim 5 wherein the visual indication is one of a color indication and a textual indication.

7. The computer-implemented method of claim 1 further comprising:
monitoring, at a user interface of a computing device, at least one sensor of a plurality of sensors from a plurality of masks; and
executing an action based upon, at least in part, the threshold range of the data.

8. A computer program product residing on a computer readable storage medium having a plurality of instructions stored thereon which, when executed across one or more processors, causes at least a portion of the one or more processors to perform operations for using a sensor incorporated into a mask comprising:
obtaining, from a sensor, data associated with a biological attribute of a user;
processing the data to determine whether the data is within a threshold range; and
providing an indication of whether the data is within the threshold range.

9. The computer program product of claim 8 wherein obtaining the data includes receiving a transmission of the data from the sensor.

10. The computer program product of claim 8 wherein the threshold range is a temperature range.

11. The computer program product of claim 8 wherein the indication an audible indication.

12. The computer program product of claim 8 wherein the indication a visual indication based upon, at least in part, the threshold range of the data.

13. The computer program product of claim 12 wherein the visual indication is one of a color indication and a textual indication.

14. The computer program product of claim 8 wherein the operations further comprise:
monitoring, at a user interface of a computing device, at least one sensor of a plurality of sensors from a plurality of masks; and
executing an action based upon, at least in part, the threshold range of the data.

15. A mask with a computing system that includes one or more processors and one or more memories configured to perform operations comprising:
obtaining, from a sensor, data associated with a biological attribute of a user;
processing the data to determine whether the data is within a threshold range; and
providing an indication of whether the data is within the threshold range.

16. The mask of claim 15 wherein obtaining the data includes receiving a transmission of the data from the sensor.

17. The mask of claim 15 wherein the threshold range is a temperature range.

18. The mask of claim 15 wherein the indication an audible indication.

19. The mask of claim 15 wherein the indication a visual indication based upon, at least in part, the threshold range of the data.

20. The mask of claim 19 wherein the visual indication is one of a color indication and a textual indication.

21. The mask of claim 15 wherein the operations further comprise:
monitoring, at a user interface of a computing device, at least one sensor of a plurality of sensors from a plurality of masks; and
executing an action based upon, at least in part, the threshold range of the data.
